(19) United States
(12) Patent Application Publication
(10) Pub. No.: US 2026/0200816 A1
(43) Pub. Date: Jul. 16, 2026

(54) MULTI-REACTOR SYSTEMS AND METHODS FOR PROPYLENE PRODUCTION

(71) Applicants: SABIC Global Technologies B.V., Bergen OP Zoom (NL); SAUDI ARABIAN OIL COMPANY (SAUDI ARAMCO), Dhahran (SA)

(72) Inventors: Zhonglin ZHANG, Dhahran (SA); Furqan ALJUMAH, Dhahran (SA); Mosab T. KHEYAMI, Dhahran (SA); Munir D KHOKHAR, Dhahran (SA); Saud ALKHUDEER, Bangalore (IN); Vidya Sagar GUGGILLA, Bangalore (IN)

(21) Appl. No.: 19/133,851

(22) PCT Filed: Nov. 28, 2023
(86) PCT No.: PCT/IB2023/061986

§ 371 Date: May 29, 2025

(30) Foreign Application Priority Data
Nov. 29, 2022 (EP) 22210382.2

(51) Int. Cl. C07C 4/06; C07C 6/04; C07C 7/04; C07C 11/06

(52) U.S. Cl. CPC C07C 4/06; C07C 6/04; C07C 7/04; C07C 11/06; C07C 2523/30; C07C 2523/36; C07C 2529/40

(57) ABSTRACT

Systems and methods for the production of propylene from C4 raffinate hydrocarbon streams. The method may include supplying a C4 raffinate hydrocarbon stream to a metathesis reactor to produce a metathesis outlet stream and separating the plurality of C5+ hydrocarbons from the propylene and the one or more C4 hydrocarbons in the metathesis outlet stream to produce a C5+ feed stream and a first propylene-rich stream. The method may further include supplying the C5+ feed stream to a cracking reactor to produce a cracked outlet stream containing propylene and the one or more C4 hydrocarbons. The method may further include separating the propylene from the one or more C4 hydrocarbons in the cracked outlet stream to produce a second propylene-rich stream and a C4-rich stream, and recycling the C4-rich stream to the metathesis reactor as part of the C4 raffinate hydrocarbon stream.

### TECHNICAL FIELD

The present disclosure generally relates to systems and methods for producing propylene from C4 hydrocarbon streams. More specifically, the present disclosure relates to, among other embodiments, systems and methods for producing propylene through the parallel operation of separate metathesis and cracking reactors.

### BACKGROUND

Propylene is a desirable industrial compound having a worldwide production greater than that of any other organic compound other than ethylene. Accordingly, propylene is the second most important starting feedstock in the petrochemical industry after ethylene. Propylene is particularly used as a feedstock for the production of polypropylene which has a wide variety of uses. Propylene is also used for the production of isopropanol, epichlorohydrin, propylene oxide, acrylonitrile, cumene, butyraldehyde, and acrylic acid, among other important chemicals. Over 85 million tons of propylene are produced worldwide each year. In addition to other production methods, propylene may be generated by the steam cracking of hydrocarbons. Accordingly, methods and systems capable of efficient propylene production from hydrocarbon feedstocks and improving feedstock utilization of hydrocarbon streams are desirable.

### SUMMARY

To address shortcomings in the art, Applicants have developed systems and methods for producing propylene from hydrocarbon streams through parallel operation of separate metathesis and cracking reactors. In at least some embodiments, the presently disclosed systems and methods may be particularly suited, to produce propylene from low cost C4 raffinate streams, that may include 1-butene, trans-2-butene, cis-2-butene, and mixtures thereof, thereby converting low value butene streams to high value propylene. The operation of separate reactors provides for, among other advantages, the use of a metathesis catalyst and a cracking catalyst under separate operating conditions, thereby maximizing propylene yield. Additionally, among other advantages, the use of a separate metathesis reactor allows for the use of low temperatures metathesis catalysts in certain embodiments and high temperature metathesis catalysts in certain other embodiments.

Methods and systems for the production of propylene are provided. In certain embodiments, the method for production of propylene may include supplying a C4 raffinate hydrocarbon stream to a metathesis reactor to produce a metathesis outlet stream. The C4 raffinate hydrocarbon stream may substantially contain one or more C4 hydrocarbons and the metathesis outlet stream may contain a plurality of C5+ hydrocarbons, propylene, and one or more C4 hydrocarbons. The method may further include separating the plurality of C5+ hydrocarbons from the propylene and the one or more C4 hydrocarbons in the metathesis outlet stream to produce a C5+ feed stream and a first propylene-rich stream. The method may also include supplying the C5+ feed stream to a cracking reactor to produce a cracked outlet stream containing propylene and the one or more C4 hydrocarbons. The method may further include separating the propylene from the one or more C4 hydrocarbons in the cracked outlet stream to produce a second propylene-rich stream and a C4-rich stream. The method may also include recycling the C4-rich stream to the metathesis reactor as part of the C4 raffinate hydrocarbon stream.

In certain embodiments, the metathesis reactor and the cracking reactor may be operated at different temperatures. For example, in certain embodiments, the metathesis reactor may be a high temperature metathesis reactor that includes a tungstate on silica catalyst and that is operated at a temperature from about 500° C. to about 550° C. In certain other exemplary embodiments, the metathesis reactor may be a low temperature metathesis reactor that includes a rhenium on alumina catalyst and that is operated at a temperature from about 50° C. to about 100° C. In certain embodiments, the cracking reactor includes a high silica ZSM-5 catalyst and is operated at a temperature from about 550° C. to about 575° C.

In certain embodiments, the method may further include supplying the C4 raffinate hydrocarbon stream to an isomerization reactor prior to supplying the C4 raffinate hydrocarbon stream to the metathesis reactor. In certain exemplary embodiments, the C4 raffinate hydrocarbon stream may include a portion of the metathesis outlet stream and a portion of the cracked outlet stream. The C4 raffinate hydrocarbon stream may include an input C4 raffinate hydrocarbon stream from a source other than the outlet streams of the metathesis reactor, the cracking reactor, and the isomerization reactor. In certain embodiments, the method may further include supplying the metathesis outlet stream to a sequence of separation columns to produce the C5+ feed stream and the first propylene-rich stream.

In certain embodiments, the method may further include supplying the cracked outlet stream to a sequence of separation columns to produce the second propylene-rich stream and the C4-rich stream. The sequence of separation columns may include, for example, a deethanizer column, a depropanizer column, and a debutanizer column. According to at least certain aspects of the present disclosure, the amount of propylene produced by combining the first propylene-rich stream and the second propylene-rich stream is in excess of 40 mol % when the C4 raffinate hydrocarbon stream contains about 70 mol % cis-2-butene and trans-2-butene and about 30 mol % n-butane.

According to certain aspects of the present disclosure, a system for the production of propylene is provided. The system may include a metathesis reactor operable to receive a C4 raffinate hydrocarbon stream and produce a metathesis outlet stream. The C4 raffinate hydrocarbon stream may substantially contain one or more C4 hydrocarbons and the metathesis outlet stream may contain a plurality of C5+ hydrocarbons, propylene, and one or more C4 hydrocarbons. The system may also contain a plurality of separation columns in fluid communication with the metathesis reactor and the cracking reactor. The plurality of separation columns may be operable to receive the metathesis outlet stream and produce a C5+ feed stream and a first propylene-rich stream. The system may also contain a cracking reactor in fluid communication with the plurality of separation columns and operable to receive the C5+ feed stream and produce a cracked outlet stream containing propylene and the one or more C4 hydrocarbons. The cracked outlet stream may be supplied to the plurality of separation columns whereby the propylene is separated from the one or more C4 hydrocarbons in the cracked outlet stream to produce a second propylene-rich stream and a C4-rich stream.

In certain embodiments, the C4-rich stream may be recycled to the metathesis reactor as part of the C4 raffinate hydrocarbon stream. The plurality of separation columns may be further operable to separate propylene from the cracked outlet stream. The plurality of separation columns may include, for example, a deethanizer column, a depropanizer column, and a debutanizer column. In certain embodiments, the system may further include an isomerization reactor in fluid communication with the metathesis reactor and the plurality of separation columns. In such embodiments, the isomerization reactor may be operable to pretreat the C4 raffinate hydrocarbon stream prior to supplying the C4 raffinate hydrocarbon stream to the metathesis reactor. In certain embodiments, the system may include an isomerization reactor in fluid communication with the metathesis reactor and the plurality of separation columns. In such embodiments, the isomerization reactor may be operable to receive one of an input C4 raffinate hydrocarbon stream or a combined C4 raffinate hydrocarbon stream formed from the combination of an input C4 raffinate hydrocarbon stream and the C4-rich stream or a portion of the cracked outlet stream containing C4 hydrocarbons. The isomerization reactor may be configured to produce an isomerized C4 raffinate hydrocarbon stream for supplying to the metathesis reactor.

In certain embodiments, the metathesis reactor and the cracking reactor may be operated at different temperatures. For example, in certain embodiments, the metathesis reactor may be a high temperature metathesis reactor that includes a tungstate on silica catalyst and that is operated at a temperature from about 500° C. to about 550° C. In certain other exemplary embodiments, the metathesis reactor may be a low temperature metathesis reactor that includes a rhenium on alumina catalyst and that is operated at a temperature from about 50° C. to about 100° C. In certain embodiments, the cracking reactor includes a high silica ZSM-5 catalyst and is operated at a temperature from about 550° C. to about 575° C. According to at least certain aspects of the present disclosure, the system may be operable to produce a propylene product distribution in excess of 40 mol % when the C4 raffinate hydrocarbon stream contains about 70 mol % cis-2-butene and trans-2-butene and about 30 mol % n-butane.

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a graphical representation of a system and method for producing propylene from a C4 raffinate hydrocarbon feed stream, according to an exemplary embodiment of the present disclosure.

FIG. 2 is a graphical representation of the simulated product yield distribution obtained using the system and method depicted in FIG. 1, according to an exemplary embodiment of the present disclosure.

### DETAILED DESCRIPTION

The present disclosure describes various embodiments related to systems and methods for producing propylene from C4 hydrocarbon streams. Further embodiments may be described and disclosed.

In the following description, numerous details are set forth in order to provide a thorough understanding of the various embodiments. In other instances, well-known processes, devices, and systems may not have been described in particular detail in order not to unnecessarily obscure the various embodiments. Additionally, illustrations of the various embodiments may omit certain features or details in order to not obscure the various embodiments.

The description may use the phrases “in some embodiments,” “in various embodiments,” “in an embodiment,” or “in embodiments,” which may each refer to one or more of the same or different embodiments. Furthermore, the terms “comprising” (and any form, such as “comprise” and “comprises”), “having” (and any form, such as “have” and “has”), “including” (and any form, such as “includes” and “include”) or“containing” (and any form, such as “contains” and “contain”) and the like, as used with respect to embodiments of the present disclosure, are synonymous and are inclusive or open-ended and do not exclude additional, unrecited elements or method steps.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “rich” with respect to a component X in a stream, when used in the claims and/or the specification, refers to component X being present in an amount greater than 50 weight percent of the totality of all components in the stream. For example, a propylene-rich stream contains propylene in an amount greater than 50 weight percent of the totality of all components in the stream. The term “substantially” with respect to a component X in a stream, when used in the claims and/or the specification, refers to component X being present in an amount greater than 90 weight percent of the totality of all components in the stream. For example, a stream containing substantially one or more C4 hydrocarbons refers to a stream containing one or more C4 hydrocarbons in an amount greater than 90 weight percent of the totality of all components in the stream.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

Disclosed here are systems and methods for producing propylene from hydrocarbon streams, particularly C4 raffinate hydrocarbon streams. In at least some embodiments, the presently disclosed systems and methods may be particularly suited, to produce propylene from low cost C4 raffinate streams, that may include 1-butene, trans-2-butene, cis-2-butene, and mixtures thereof, thereby converting low value butene streams to high value propylene. According to certain aspects of the present disclosure, the presently disclosed systems and methods include parallel operation of separate metathesis and cracking reactors which provides for, among other advantages, the use of a metathesis catalyst and a cracking catalyst under separate operating conditions, thereby maximizing propylene yield. Additionally, among other advantages, the use of a separate metathesis reactor allows for the use of low temperatures metathesis catalysts in certain embodiments and high temperature metathesis catalysts in certain other embodiments. According to certain aspects of the present disclosure, an advantage of the presently disclosed methods and systems having separate metathesis and cracking reactors is that the outlet stream of the metathesis reactor may be fed to one or more separator units prior to feeding the stream to cracking reactor, thereby enhancing the cracking efficiency and providing an overall yield approaching 99%. By contrast, systems and methods that carry out metathesis and cracking in a single unit, such as a dual bed reactor, provides only approximately a 96% yield. In the single reactor, the overall yield of propylene is compensated with little formation of propane in the final product slate, thereby decreasing the propylene yield by approximately 3%. Decoupling the metathesis and cracking reactors offers the advantage of operating the metathesis catalysts and the cracking catalysts at their optimal temperatures to maximize yield as well as lifetimes. It also allows provides the option to optimize recycle streams and operate the reactors at the higher conversion possible. In at least some embodiments, the cracking reactor is configured to only process C5+ hydrocarbons, particularly the C5+ heavy hydrocarbons produced from the metathesis reaction. In at least some embodiments, the metathesis reactor is configured to only process C4 hydrocarbon feed streams.

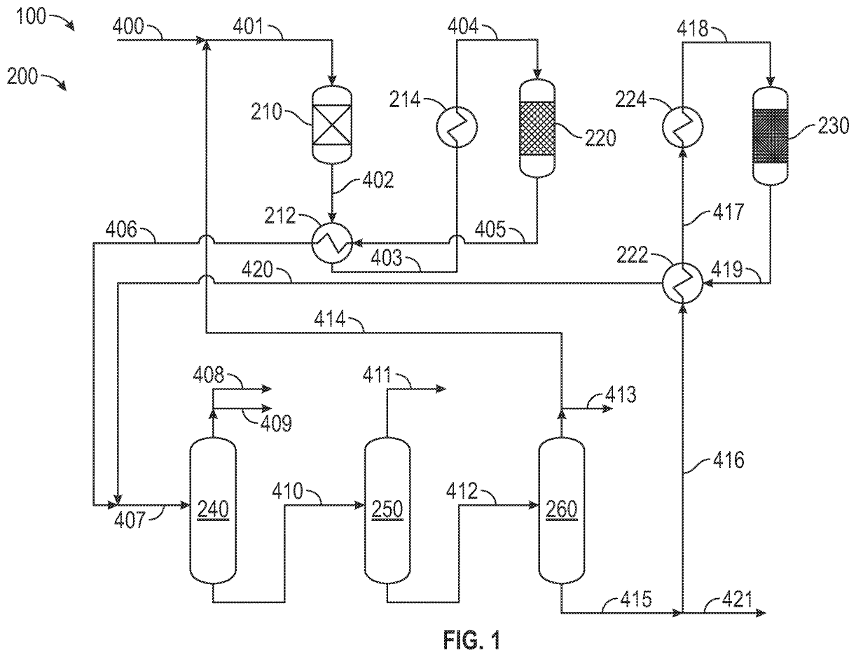

FIG. 1 depicts a method 100 and a system 200 for producing propylene from a C4 raffinate hydrocarbon feed stream 400, according to an exemplary embodiment of the present disclosure. Other embodiments of the presently disclosed systems and methods are possible. As depicted in the exemplary embodiment provided in FIG. 1, method 100 may include supplying C4 raffinate feed stream 400 to a pretreatment reactor such as isomerization reactor 210 via C4 raffinate pretreatment stream 401. The C4 raffinate feed stream may include, for example, 1-butene, trans-2-butene, cis-2-butene, and mixtures thereof. In certain embodiments, the isomerization reactor may include a palladium on alumina catalyst. The isomerization reactor is operable to isomerize the C4 raffinate feed stream and any other C4 recycle stream 414 in the form of C4 raffinate pretreatment stream 401 to produce isomerized C4 stream 402 by converting butenes to equilibrium. Isomerized C4 stream 402 may be optionally supplied to a metathesis preheater 212 to produce a preheated C4 stream 403. Preheated C4 stream 403 may then be supplied to metathesis heater 214 to generate a heated C4 stream 404 or metathesis reactor inlet stream 404. Heated C4 stream/metathesis reactor inlet stream 404 may then be fed to metathesis reactor 220 to generate metathesis reactor outlet stream 405.

Metathesis reactor outlet stream 405 may be optionally fed to preheater 212 before being fed to a plurality of separation columns 240, 250, 260. In particular, metathesis reactor outlet stream 405 may be supplied to preheater 212 to form deethanizer feed from metathesis reactor stream 406. Deethanizer feed from metathesis reactor stream 406 may be combined with deethanizer feed from cracking reactor stream 420 to form deethanizer column feed stream 407 which may in turn be supplied to deethanizer column 240. Deethanizer column 240 is operable to receive the deethanizer column feed stream 407 and generate ethylene 409 in addition to light gas purge 408 and deethanizer outlet stream/depropanizer column feed 410. Depropanizer column feed 410 may then be supplied to depropanizer column 250 to generate propylene 411 and depropanizer outlet stream/debutanizer column feed 412. Debutanizer column feed 412 may then be supplied to debutanizer column 260 to generate C4 recycle stream 414, C4 purge stream 413, and debutanizer column bottom stream 415. C4 recycle stream 414 may be combined with C4 raffinate feed stream 400 to form C4 pretreatment stream 401 which may in turn be supplied to isomerization reactor 210 and metathesis reactor 220.

The debutanizer column bottom stream 415 may be supplied to cracking reactor preheater 222 in the form of C5+ recycle stream 416 following potential split from C5+ purge stream 421. As used herein, the term “C5+” or “C5+ hydrocarbons,” refers to hydrocarbons having five (5) or more carbon atoms. Similar terms, such as “C3+ hydrocarbons,” “C4+ hydrocarbons,” and “C6+ hydrocarbons,” also refers to hydrocarbons having three (3) or more carbon atoms, four (4) or more carbons atoms, and six (6) or more carbon atoms, respectively. Cracking reactor preheater 222 is operable to preheat C5+ recycle stream 416 to produce preheated C5+ recycle stream 417. Preheated C5+ recycle stream 417 may be supplied to cracking reactor heater 224 to produce heated C5+ recycle stream/cracking reactor inlet stream 418. Cracking reactor inlet stream 418 may be supplied to cracking reactor 230 to generate cracking reactor outlet stream 419. Cracking reactor outlet stream 419 may be preheated at cracking reactor preheater 222 to generate deethanizer feed from cracking reactor stream 420, which may in turn be combined with deethanizer feed from metathesis reactor stream 406 to form deethanizer column feed stream 407 for supply to dethanizer column 240.

As depicted in FIG. 1, method 100 may include supplying a C4 raffinate hydrocarbon stream 400, 401, 404 to a metathesis reactor 220 to produce a metathesis outlet stream 405. The C4 raffinate hydrocarbon stream 400, 401 may substantially contain one or more C4 hydrocarbons and the metathesis outlet stream 405 may containing a plurality of C5+ hydrocarbons, propylene, and one or more C4 hydrocarbons. Method 100 may further include separating the plurality of C5+ hydrocarbons from the propylene and the one or more C4 hydrocarbons in the metathesis outlet stream 405, 406, for example via supply to the plurality of separation columns 240, 250, 260, to produce a C5+ feed stream 415, 416 and a first propylene-rich stream 411. Method 100 may further include supplying the C5+ feed stream 415, 416 to a cracking reactor 230 to produce a cracked outlet stream 419 containing propylene and the one or more C4 hydrocarbons. Method 100 may further include separating the propylene from the one or more C4 hydrocarbons in the cracked outlet stream 419 to produce a second propylene-rich stream 411 and a C4-rich stream 414. Method 100 may further include recycling the C4-rich 414 stream to the metathesis reactor 220 as part of the C4 raffinate hydrocarbon stream 401. Method 100 may further include supplying the C4 raffinate hydrocarbon stream 400 to an isomerization reactor 210 prior to supplying the C4 raffinate hydrocarbon stream 400 to the metathesis reactor 220.

In certain embodiments of method 100, the C4 raffinate hydrocarbon stream 400, 401 may include a portion of the metathesis outlet stream 405 and a portion of the cracked outlet stream 419 in the form of C4 recycle stream 414. In certain embodiments, the C4 raffinate hydrocarbon stream, such as C4 pretreatment stream 401 may include an input C4 raffinate hydrocarbon stream from a source other than the outlet streams of the metathesis reactor 220, the cracking reactor 230, and the isomerization reactor 210, such as fresh C4 raffinate feed stream 400. The metathesis outlet stream 405 may be supplied to a sequence of separation columns to produce the C5+ feed stream 416, 417, 418 and the first propylene-rich stream 411. Method 100 may further include supplying the cracked outlet stream 419, 420 to a sequence of separation columns 240, 250, 260 to produce the second propylene-rich stream 411 and the C4-rich stream 414. The sequence of separation columns may include a deethanizer column 240, a depropanizer column 250, and a debutanizer column 260. In certain embodiments, method 100 may produce a propylene product distribution in excess of 40 mol % when the C4 raffinate hydrocarbon stream contains about 70 mol % cis-2-butene and trans-2-butene and about 30 mol % n-butane.

As depicted in FIG. 1, system 200 may include a metathesis reactor 220 operable to receive a C4 raffinate hydrocarbon stream 400, 401 and produce a metathesis outlet stream 405. The C4 raffinate hydrocarbon stream 400, 401 may substantially contain one or more C4 hydrocarbons and the metathesis outlet stream 405 may contain a plurality of C5+ hydrocarbons, propylene, and one or more C4 hydrocarbons. System 200 may also include a plurality of separation columns, such as separation columns 240, 250, 260 in fluid communication with the metathesis reactor 220 and the cracking reactor 230. The plurality of separation columns 240, 250, 260 may be operable to receive the metathesis outlet stream 405, 406, 407 and produce a C5+ feed stream 416, 417, 418 and a first propylene-rich stream 411. System 200 may also include a cracking reactor 230 in fluid communication with the plurality of separation columns 240, 250, 260 and operable to receive the C5+ feed stream 416, 417, 418 and produce a cracked outlet stream 419 containing propylene and the one or more C4 hydrocarbons. The system 200 may also be configured to supply the cracked outlet stream 419 to the plurality of separation columns 240, 250, 260 such that propylene is separated from the one or more C4 hydrocarbons in the cracked outlet stream to produce a second propylene-rich stream 411 and a C4-rich stream 414. The C4-rich stream 414 may be recycled to the metathesis reactor 220 as part of the C4 raffinate hydrocarbon stream 401, such as C4 raffinate pretreatment stream 401. The plurality of separation columns 240, 250, 260 may be further operable to separate propylene from the cracked outlet stream 419, 420, 407. The plurality of separation columns may include a deethanizer column 240, a depropanizer column 250, and a debutanizer column 260.

System 200 may further include an isomerization reactor 210 in fluid communication with the metathesis reactor 220 and the plurality of separation columns 240, 250, 260. The isomerization reactor may be operable to pretreat the C4 raffinate hydrocarbon stream 400, 401 prior to supplying the C4 raffinate hydrocarbon stream 404 to the metathesis reactor 220. The isomerization reactor 210 may be operable to receive one of an input C4 raffinate hydrocarbon stream 400 or a combined C4 raffinate hydrocarbon stream 401 formed from the combination of an input C4 raffinate hydrocarbon stream 400 and the C4-rich stream 414 and/or a portion of the cracked outlet stream containing C4 hydrocarbons 419, 414. The isomerization reactor 210 may also be configured to produce an isomerized C4 raffinate hydrocarbon stream 402 for supplying to the metathesis reactor 220.

In certain embodiments of system 200, the metathesis reactor 220 and the cracking reactor 230 may be fixed bed reactors. In certain embodiments, the metathesis reactor 220 and the cracking reactor 230 may be operated at different temperatures. For example, in certain embodiments, the metathesis reactor 220 may be a high temperature metathesis reactor 220 that includes a tungstate on silica catalyst and that is operated at a temperature from about 500° C. to about 550° C. In certain other exemplary embodiments, the metathesis reactor 220 may be a low temperature metathesis reactor 220 that includes a rhenium on alumina catalyst and that is operated at a temperature from about 50° C. to about 100° C. In certain embodiments, the cracking reactor 230 includes a high silica ZSM-5 catalyst and is operated at a temperature from about 550° C. to about 575° C. According to at least certain aspects of the present disclosure, the system 200 may be operable to produce a propylene product distribution in excess of 40 mol % when the C4 raffinate hydrocarbon stream contains about 70 mol % cis-2-butene and trans-2-butene and about 30 mol % n-butane.

### EXAMPLES

The examples provided below illustrate selected aspects of the various methods and systems for producing propylene from C4 raffinate hydrocarbon streams.

### Example 1

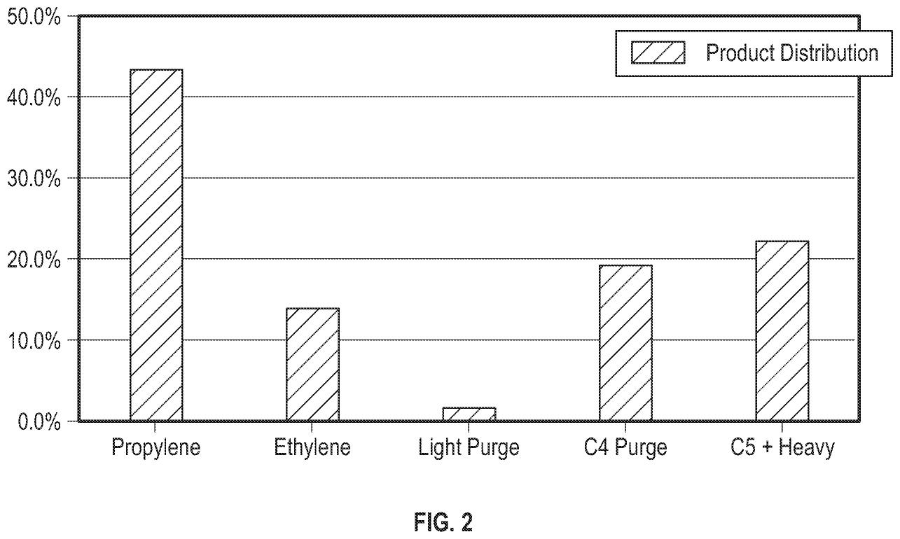

Production yields for method 100 and system 200 depicted in FIG. 1 are shown in Table 1 as determined by simulation using an AspenPlus10 simulator based on the use of a W/Si catalyst and a C4 raffinate feed stream containing a mixture of 30 mol % n-butane and 70 mol % cis-2-butene and trans-2-butene. The simulated product yield distributions are provided in Tables 1 and 2 and FIG. 2.

TABLE 1
Material Balance in Weight %

| Stream | 400 | 404 | 405 | 407 | 408 | 409 | 410 | 411 | 412 | 413 | 414 | 415 | 418 | 420 | 421 |
|---|---|---|---|---|---|---|---|---|---|---|---|---|---|---|---|
| Mol Flow | 100 | 268 | 270 | 406 | 1.6 | 14.1 | 390 | 63.2 | 327 | 29.7 | 168 | 129 | 116 | 136 | 12.9 |
| (kmol/hr) | | | | | | | | | | | | | | | |
| Mass Flow | 5.67 | 15.3 | 15.3 | 23.1 | 0.04 | .394 | 22.7 | 2.66 | 20.0 | 1.70 | 9.61 | 8.73 | 7.86 | 7.86 | 0.87 |
| (103 kg/hr) | | | | | | | | | | | | | | | |
| MW | 56.7 | 56.9 | 56.6 | 57.0 | 27.9 | 27.9 | 58.2 | 42.1 | 61.3 | 57.1 | 57.1 | 67.8 | 67.8 | 57.9 | 67.8 |
| Methane | 0.0 | 0.0 | 0.0 | 0.0 | 3.1 | 3.1 | 0.0 | 0.0 | 0.0 | 0.0 | 0.0 | 0.0 | 0.0 | 0.2 | 0.0 |
| Ethylene | 0.0 | 0.0 | 0.6 | 1.7 | 90.0 | 90.0 | 0.0 | 0.0 | 0.0 | 0.0 | 0.0 | 0.0 | 0.0 | 3.8 | 0.0 |
| Ethane | 0.0 | 0.0 | 0.0 | 0.0 | 2.1 | 2.1 | 0.0 | 0.0 | 0.0 | 0.0 | 0.0 | 0.0 | 0.0 | 0.1 | 0.0 |
| Propylene | 0.0 | 0.0 | 9.2 | 11.2 | 4.7 | 4.7 | 11.3 | 96.1 | 0.0 | 0.0 | 0.0 | 0.0 | 0.0 | 15.0 | 0.0 |
| Propane | 0.0 | 0.0 | 0.0 | 0.4 | 0.0 | 0.0 | 0.4 | 3.8 | 0.0 | 0.0 | 0.0 | 0.0 | 0.0 | 1.3 | 0.0 |
| 1-Butene | 0.0 | 9.0 | 9.7 | 7.8 | 0.0 | 0.0 | 7.9 | 0.0 | 9.0 | 14.3 | 14.3 | 2.1 | 2.1 | 3.9 | 2.1 |
| Isobutene | 0.0 | 15.2 | 15.2 | 12.9 | 0.0 | 0.0 | 13.2 | 0.0 | 14.9 | 24.2 | 24.2 | 2.9 | 2.9 | 8.5 | 2.9 |
| cis-2- | 35.0 | 19.7 | 8.8 | 7.6 | 0.0 | 0.0 | 7.7 | 0.0 | 8.8 | 10.7 | 10.7 | 6.3 | 6.3 | 5.3 | 6.3 |
| Butene | | | | | | | | | | | | | | | |
| trans-2- | 35.0 | 20.1 | 5.8 | 7.3 | 0.0 | 0.0 | 7.4 | 0.0 | 8.4 | 11.3 | 11.3 | 4.7 | 4.7 | 10.1 | 4.7 |
| Butene | | | | | | | | | | | | | | | |
| Butanes | 30.0 | 34.7 | 34.7 | 24.2 | 0.0 | 0.0 | 24.7 | 0.0 | 28.0 | 37.5 | 37.5 | 15.7 | 15.7 | 3.9 | 15.7 |
| Pentenes | 0.0 | 1.3 | 15.0 | 20.5 | 0.0 | 0.0 | 20.9 | 0.0 | 23.7 | 2.1 | 2.1 | 51.6 | 51.6 | 31.1 | 51.6 |
| Hexenes | 0.0 | 0.0 | 1.0 | 3.1 | 0.0 | 0.0 | 3.2 | 0.0 | 3.6 | 0.0 | 0.0 | 8.3 | 8.3 | 7.4 | 8.3 |
| Heptenes | 0.0 | 0.0 | 0.0 | 3.2 | 0.0 | 0.0 | 3.3 | 0.0 | 3.7 | 0.0 | 0.0 | 8.5 | 8.5 | 9.4 | 8.5 |
| Total Wt % | 100 | 100 | 100 | 100 | 100 | 100 | 100 | 100 | 100 | 100 | 100 | 100 | 100 | 100 | 100 |

TABLE 2
Material Balance in Mol %

| Stream | 400 | 404 | 405 | 407 | 408 | 409 | 410 | 411 | 412 | 413 | 414 | 415 | 418 | 420 | 421 |
|---|---|---|---|---|---|---|---|---|---|---|---|---|---|---|---|
| Mol Flow | 100 | 268 | 270 | 406 | 1.6 | 14.1 | 390 | 63.2 | 327 | 29.7 | 168 | 129 | 116 | 136 | 12.9 |
| (kmol/hr) | | | | | | | | | | | | | | | |
| Mass Flow | 5.67 | 15.3 | 15.3 | 23.1 | 0.04 | .394 | 22.7 | 2.66 | 20.0 | 1.70 | 9.61 | 8.73 | 7.86 | 7.86 | 0.87 |
| (103 kg/hr) | | | | | | | | | | | | | | | |
| MW | 56.7 | 56.9 | 56.6 | 57.0 | 27.9 | 27.9 | 58.2 | 42.1 | 61.3 | 57.1 | 57.1 | 67.8 | 67.8 | 57.9 | 67.8 |
| Methane | 0.0 | 0.0 | 0.0 | 0.2 | 5.4 | 5.4 | 0.0 | 0.0 | 0.0 | 0.0 | 0.0 | 0.0 | 0.0 | 0.6 | 0.0 |
| Ethylene | 0.0 | 0.0 | 1.3 | 3.5 | 89.5 | 89.5 | 0.0 | 0.0 | 0.0 | 0.0 | 0.0 | 0.0 | 0.0 | 7.8 | 0.0 |
| Ethane | 0.0 | 0.0 | 0.0 | 0.0 | 1.9 | 1.9 | 0.0 | 0.1 | 0.0 | 0.0 | 0.0 | 0.0 | 0.0 | 0.3 | 0.0 |
| Propylene | 0.0 | 0.0 | 12.3 | 15.1 | 3.1 | 3.1 | 15.6 | 96.3 | 0.0 | 0.0 | 0.0 | 0.0 | 0.0 | 20.7 | 0.0 |
| Propane | 0.0 | 0.0 | 0.0 | 0.6 | 0.0 | 0.0 | 0.6 | 3.6 | 0.0 | 0.0 | 0.0 | 0.0 | 0.0 | 1.7 | 0.0 |
| 1-Butene | 0.0 | 9.1 | 9.8 | 7.9 | 0.0 | 0.0 | 8.2 | 0.0 | 9.8 | 14.5 | 14.5 | 2.5 | 2.5 | 4.0 | 2.5 |
| Isobutene | 0.0 | 15.4 | 15.3 | 13.1 | 0.0 | 0.0 | 13.7 | 0.0 | 16.3 | 24.6 | 24.6 | 3.5 | 3.5 | 8.7 | 3.5 |
| cis-2- | 35.4 | 20.0 | 8.8 | 7.7 | 0.0 | 0.0 | 8.0 | 0.0 | 9.6 | 10.8 | 10.8 | 7.6 | 7.6 | 5.5 | 7.6 |
| Butene | | | | | | | | | | | | | | | |
| trans-2- | 35.4 | 20.4 | 5.9 | 7.4 | 0.0 | 0.0 | 7.7 | 0.0 | 9.2 | 11.5 | 11.5 | 5.7 | 5.7 | 10.4 | 5.7 |
| Butene | | | | | | | | | | | | | | | |
| Butanes | 29.3 | 34.0 | 33.8 | 23.8 | 0.0 | 0.0 | 24.7 | 0.0 | 29.5 | 36.8 | 36.8 | 18.3 | 18.3 | 3.9 | 18.3 |
| Pentenes | 0.0 | 1.1 | 12.1 | 16.7 | 0.0 | 0.0 | 17.3 | 0.0 | 20.7 | 1.7 | 1.7 | 49.8 | 49.8 | 25.7 | 49.8 |
| Hexenes | 0.0 | 0.0 | 0.6 | 2.1 | 0.0 | 0.0 | 2.2 | 0.0 | 2.6 | 0.0 | 0.0 | 6.7 | 6.7 | 5.1 | 6.7 |
| Heptenes | 0.0 | 0.0 | 0.0 | 1.9 | 0.0 | 0.0 | 1.9 | 0.0 | 2.3 | 0.0 | 0.0 | 5.8 | 5.8 | 5.6 | 5.8 |
| Total | 100 | 100 | 100 | 100 | 100 | 100 | 100 | 100 | 100 | 100 | 100 | 100 | 100 | 100 | 100 |
| Mol % | | | | | | | | | | | | | | | |

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A method for production of propylene, the method comprising:
supplying a C4 raffinate hydrocarbon stream to a metathesis reactor to produce a metathesis outlet stream, the C4 raffinate hydrocarbon stream containing substantially one or more C4 hydrocarbons and the metathesis outlet stream containing a plurality of C5+ hydrocarbons, propylene, and one or more C4 hydrocarbons;
separating the plurality of C5+ hydrocarbons from the propylene and the one or more C4 hydrocarbons in the metathesis outlet stream to produce a C5+ feed stream and a first propylene-rich stream;
supplying the C5+ feed stream to a cracking reactor to produce a cracked outlet stream containing the propylene and the one or more C4 hydrocarbons;
separating the propylene from the one or more C4 hydrocarbons in the cracked outlet stream to produce a second propylene-rich stream and a C4-rich stream; and
recycling the C4-rich stream to the metathesis reactor as part of the C4 raffinate hydrocarbon stream.

2. The method according to claim 1, wherein the metathesis reactor and the cracking reactor are operated at different temperatures.

3. The method according to claim 1, wherein the metathesis reactor is a high temperature metathesis reactor comprising a tungstate on silica catalyst and operated at a temperature from about 500° C. to about 550° C.

4. The method according to claim 1, wherein the metathesis reactor is a low temperature metathesis reactor comprising a rhenium on alumina catalyst and operated at a temperature from about 50° C. to about 100° C.

5. The method according to claim 1, wherein the cracking reactor comprises a high silica ZSM-5 catalyst and is operated at a temperature from about 550° C. to about 575° C.

6. The method according to claim 1, further comprising:
supplying the C4 raffinate hydrocarbon stream to an isomerization reactor prior to supplying the C4 raffinate hydrocarbon stream to the metathesis reactor.

7. The method according to claim 1, wherein the C4 raffinate hydrocarbon stream comprises a portion of the metathesis outlet stream and a portion of the cracked outlet stream.

8. The method according to claim 1, wherein the metathesis outlet stream is supplied to a sequence of separation columns to produce the C5+ feed stream and the first propylene-rich stream.

9. The method according to claim 1, wherein the cracked outlet stream is supplied to a sequence of separation columns to produce the second propylene-rich stream and the C4-rich stream.

10. The method according to claim 1, wherein the C4 raffinate hydrocarbon stream contains one or more of cis-2-butene, trans-2-butene, and butane.

11. The method according to claim 1, wherein an amount of propylene produced by combining the first propylene-rich stream and the second propylene-rich stream is in excess of 40 mol % when the C4 raffinate hydrocarbon stream contains about 70 mol % cis-2-butene and trans-2-butene and about 30 mol % n-butane.

12. A system for production of propylene, the system comprising:
a metathesis reactor operable to receive a C4 raffinate hydrocarbon stream and produce a metathesis outlet stream, the C4 raffinate hydrocarbon stream containing substantially one or more C4 hydrocarbons and the metathesis outlet stream containing a plurality of C5+ hydrocarbons, propylene, and one or more C4 hydrocarbons;
a plurality of separation columns in fluid communication with the metathesis reactor and a cracking reactor, the plurality of separation columns operable to receive the metathesis outlet stream and produce a C5+ feed stream and a first propylene-rich stream; and
the cracking reactor in fluid communication with the plurality of separation columns and operable to receive the C5+ feed stream and produce a cracked outlet stream containing the propylene and the one or more C4 hydrocarbons, the cracked outlet stream being supplied to the plurality of separation columns whereby the propylene is separated from the one or more C4 hydrocarbons in the cracked outlet stream to produce a second propylene-rich stream and a C4-rich stream.

13. The system according to claim 12, wherein the C4-rich stream is recycled to the metathesis reactor as part of the C4 raffinate hydrocarbon stream; and wherein the plurality of separation columns comprises a deethanizer column, a depropanizer column, and a debutanizer column.

14. The system according to claim 12,
wherein the metathesis reactor is a high temperature metathesis reactor comprising a tungstate on silica catalyst and operated at a temperature from about 500° C. to about 550° C., or the metathesis reactor is a low temperature metathesis reactor comprising a rhenium on alumina catalyst and operated at a temperature from about 50° C. to about 100° C.; and
wherein the cracking reactor comprises a high silica ZSM-5 catalyst and is operated at a temperature from about 550° C. to about 575° C.

15. The system according to claim 12, further comprising:
an isomerization reactor in fluid communication with the metathesis reactor and the plurality of separation columns, the isomerization reactor operable to receive one of the input C4 raffinate hydrocarbon stream or a combined C4 raffinate hydrocarbon stream formed from the combination of the input C4 raffinate hydrocarbon stream and the C4-rich stream or a portion of the cracked outlet stream comprising C4 hydrocarbons, the isomerization reactor configured to produce an isomerized C4 raffinate hydrocarbon stream for supplying to the metathesis reactor.
